(19) United States
(12) Patent Application Publication
(10) Pub. No.: US 2010/0000549 A1
(43) Pub. Date: Jan. 7, 2010

(54) MATERIAL, CUSHION AND SUPPORT FOR IMMOBILISING A SUBJECT FOR MEDICAL TREATMENT

(75) Inventor: Reginald Nieberding, Putte (NL)

Correspondence Address: KNOBBE MARTENS OLSON & BEAR LLP, 2040 MAIN STREET, FOURTEENTH FLOOR, IRVINE, CA, 92614, US

(73) Assignee: T-Tape Company BV, Putte (NL)

(21) Appl. No.: 12/495,172

(22) Filed: Jun. 30, 2009

(30) Foreign Application Priority Data
Jul. 2, 2008 (EP) 08159536.5

(51) Int. Cl. A61N 5/00

(52) U.S. Cl. 128/845; 128/869

(57) ABSTRACT

A flexible thermoplastic sheet material (100) is disclosed, suitable for molding to and immobilising a part of a subject during medical treatment. The flexible thermoplastic sheet material has a core layer (10) with an upper surface (1) and lower surface (2), that is a thermoplastic composition including polycaprolactone and polyurethane. A first outer layer (5) is disposed over the upper surface of the core layer, having a material formed from a yarn which includes polyamide and elastane. The second outer layer (15) is disposed over the lower surface of the core layer and includes open cell foam with layers which are bonded so as to form a single sheet. A cushion and molding frame can be made from the sheet material (100).

The present invention concerns a material and device for supporting and immobilising a portion of a subject for medical treatment. In particular it concerns a device for immobilising the head and neck, or other parts of the body of a subject for radiotherapy treatment.

### BACKGROUND TO THE INVENTION

Patents being treated by radiotherapy for a tumourous growth are typically required to receive radiation doses at regular intervals, whereby each dose is precisely directed to the same location of the body. This necessitates securing devices that are adapted to the shape of the body portion being treated, which helps accurately and quickly to align the radiation source with the location of the tumour.

Commonly, tables or chairs at the radiation source, e.g., linear accelerator, CT machine, etc., support the patient in a prone or supine position while the relevant portion of the patient's body is held in a fixed or immobilised condition. The aforementioned securing device immobilises the subject by mounting the body part on a patient support table where treatment is delivered.

For example, the treatment of cancer to the head, neck or throat entails that a rigid mask molded to conform to the contours of the patient's face, is placed over the subject's face that immobilises the head, which mask precisely locates onto a patient support table whose position relative to the source is calibrated. The head of the subject may be placed accurately and repeatedly, owing to the mask which immobilises the head relative to the source.

Known the art is a device for immobilising the head of a subject that comprises a mask of thermoplastic sheet material molded to fit the face, and a second mask molded for fit the back of the head. The person who is to undergo radiotherapy is first placed in a prone position and a first thermoplastic material sheet is placed over the back of his head and neck. The subject's head is pushed onto a cushion and the thermoplastic material sheet is allowed to conform to the contours of the back portion of his head and neck. Once the first thermoplastic sheet has been conformed and has hardened, the subject is then placed in a supine position and the first sheet is inverted such that his head neck is supported by it. A second thermoplastic sheet or mask is then used to conform to the contours of the subject's face. After the face mask has hardened the two rigid masks can then be used as a unit to hold and immobilize the subject's head and neck for radiation therapy or any other purpose.

The problems in art concern the degree of physiological and psychological discomfort experienced by the patient during the molding process. Subjects report feeling of claustrophobia, in addition to undesirable reactions to the materials used to prepare the masks. There is a need in the art for a thermoplastic material that provides more comfort to the subject, and for a system that expedites the molding process. The present invention provides a new material for preparing a mask that provides more comfort without sacrificing the requisite immobilisation qualities, and also a device that serves both to expedite molding and secure the subject to the treatment table. It further provides an inflatable cushion made from the new material that allows a mold to be taken more reliably while maximizing comfort to the subject. The cushion is particularly effective at avoiding false impressions that arise when deep impressions are taken and the thermoplastic material contacts and becomes limited by the dimensions of the molding device, thereby giving an incorrect impression.

### SUMMARY OF THE INVENTION

One embodiment of the invention is a flexible thermoplastic sheet material (100), suitable for molding to and immobilising a part of a subject during medical treatment, comprising:
a core layer (10) having an upper surface (1) and lower surface (2), that is a thermoplastic composition comprising polycaprolactone and polyurethane,
a first outer layer (5) disposed over the upper surface of the core layer, comprising a material formed from a yarn comprising polyamide and elastane,
a second outer layer (15) disposed over the lower surface of the core layer comprising open cell foam,

which layers bonded so as to form a single sheet.

Another embodiment of the invention is a flexible thermoplastic sheet material (100) as described above, wherein the core layer (10) comprises 20% to 40%, polyurethane, and 60% to 80% (w/w) polycaprolactone.

Another embodiment of the invention is a flexible thermoplastic sheet material (100) as described above, wherein the core layer (10) further comprises between 1 to 40% (w/w) of non-metallic, heat-accumulating microspheres.

Another embodiment of the invention is a flexible thermoplastic sheet material (100) as described above, wherein the yarn of the first outer layer (5) comprises between 80% to 95% polyamide, and between 5% and 15% elastane.

Another embodiment of the invention is a flexible thermoplastic sheet material (100) as described above, wherein the fabric weight of the first outer layer (5) is between 210 g/m² and 230 g/m².

Another embodiment of the invention is a flexible thermoplastic sheet material (100) as described above, wherein the thickness of the first outer layer (5) is between 0.05 and 1.5 mm.

Another embodiment of the invention is a flexible thermoplastic sheet material (100) as described above, wherein the second outer layer (15) is made from polyurethane, polyester polyurethane or polyether open-cell foam.

Another embodiment of the invention is a flexible thermoplastic sheet material (100) as described above, further comprising an intervening layer (12, 12′) disposed between the core layer (10) and the first outer layer (5), and/or disposed between the core layer (10) and the second outer layer (15), made from the same material as the core (10) and with a higher polycaprolactone content.

Another embodiment of the invention is a flexible thermoplastic sheet material (100) as described above, having a maximum total thickness of 1.5 to 1.7 mm.

Another embodiment of the invention is an inflatable cushion (300) dimensioned to receive a body portion comprising
a first flexible sheet (110) made at least partially from a thermoplastic sheet (100) as described above
a second flexible sheet (115), optionally made at least partially from a thermoplastic sheet as described above,

which sheets are sealed together so as form a lumen (117) that can receive and retain inflation medium, and
an inflation nozzle (140) for the passage of inflation medium to the lumen (117).

Another embodiment of the invention is an inflatable cushion (300) as describe above, further comprising one or more side pleats (130) between the first (110) and second (115) flexible sheets.

Another embodiment of the invention is an inflatable cushion (300) as describe above, further comprising an attachment means (120, 125, 143, 145) for attachment of the cushion to a molding frame (300).

Another embodiment of the invention is an inflatable cushion (300) as describe above, formed from a single piece of thermoplastic sheet (100) as described above, folded and sealed so as form the lumen (117).

Another embodiment of the invention is a molding frame (200) having a top end (67) and a base end (69) comprising two rigid longitudinal supports (50, 52) arranged opposite each other flanking an opening (56) and configured to receive a sheet (200) as described above, or inflatable cushion (300) as described above over the top end (67) of the opening (56).

Another embodiment of the invention is a molding frame (200) as described above, wherein each longitudinal support (50, 52) comprises at the base end one or more securing means (70, 70′) adapted for demountable attachment to a treatment table or to a rigid plate that couples to the treatment table.

Another embodiment of the invention is a molding frame (200) as described above, wherein the depth, D, of the opening (56) varies along the length of each longitudinal support (50, 52).

Another embodiment of the invention is a molding frame (200) as described above, wherein the depth, D, of the opening (56) is greater at the second end (68) than at the first end (66).

Another embodiment of the invention is an inflatable cushion (300) comprising
a first flexible sheet (110) made at least partially from a sheet (100) as described above wherein the first outer layer is optional,
a second flexible sheet (115), optionally made at least partially from a sheet as described above wherein the first outer layer is optional,

which sheets are sealed together so as form a lumen (117) that can receive and retain inflation medium, and
an inflation nozzle (140) for the passage of inflation medium to the lumen (117).

Another embodiment of the invention is an inflatable cushion (300) as described above, further comprising one or more side pleats (130) between the first (110) and second (115) flexible sheets.

Another embodiment of the invention is an inflatable cushion (300) as described above, formed from a single piece of thermoplastic sheet (100) as described above, folded and sealed so as form the lumen (117).

### FIGURE LEGENDS

FIG. 1 depicts a schematic drawing of a cross section through a sheet of thermoplastic material according to the invention.

FIG. 2 depicts a schematic drawing of a cross section through a sheet of thermoplastic material according to the invention, further comprising intervening layers.

FIG. 3A depicts a plan view of a sheet of the invention after molding to the back of a head and neck of a subject, whereby contour lines show the relief of the impression.

FIG. 3B depicts a cross section of the sheet of FIG. 3A.

FIG. 4 shows a plan view of sheet of the invention, disposed with attachment means.

FIG. 5 shows perspective view of an inflatable cushion of the invention disposed with a nozzle at a folded end.

FIG. 6 shows perspective view of an inflatable cushion of the invention disposed with a nozzle at a side pleat.

FIG. 7A shows perspective view of a molding frame of the invention.

FIG. 7B shows a transverse (A-A′) cross section through a longitudinal support of FIG. 7A.

FIG. 8 shows perspective view of a molding frame of the invention disposed with a sheet of the invention.

FIG. 9A shows perspective view of a molding frame of the invention disposed with an inflatable cushion of the invention.

FIG. 9B shows a transverse cross section through a longitudinal support of FIG. 8A.

FIG. 10 shows perspective view of a molding frame of the invention wherein the depth of the opening at the second end 68 is greater than that at the first end 66.

FIG. 11A shows a perspective view of a cushion of the invention attached to a pair of buffering structures.

FIG. 11B shows a transverse (B-B′) cross section through the buffering structures and cushion of FIG. 10A.

FIG. 12 shows perspective view of a molding frame of the invention attached by a buffering structure to a cushion of the invention.

FIG. 13 shows a transverse cross section through the supports and inflated cushion and of FIG. 9, and the body part to be applied.

FIG. 14 shows a transverse cross section through the supports and inflated cushion and of FIG. 9 after the application of the body part.

FIG. 15 shows a transverse cross section through the buffering structures and cushion of the invention, depicting a variation of the buffering structures.

FIG. 16 shows a transverse cross section through the buffering structures and cushion of the invention, depicting a further variation of the buffering structures.

FIG. 17A shows a plan view of a bed frame disposed with rails to receive a cushion of the invention.

FIG. 17B shows a side view along the first end of a bed frame of FIG. 17A disposed with rails to receive a cushion of the invention.

FIG. 18A shows a plan view of a frame according to the invention, FIG. 18B shows the side elevation and FIG. 18C shows a front elevation.

### DETAILED DESCRIPTION OF THE INVENTION

The present invention provides a thermoplastic sheet material 100, suitable for molding to and immobilising a part of a subject during medical treatment, comprising:
a core layer 10 having an first (e.g. upper) surface 1 and a second (e.g. lower) surface 2, that is a thermoplastic composition comprising polycaprolactone and polyurethane,
a first outer layer 5 disposed over the upper surface of the core layer, formed from a yarn comprising polyamide and elastane, and
a second outer layer 15 disposed over the lower surface of the core layer comprising open cell foam,

which layers bonded so as to form a single sheet.

The sheet exhibits excellent deformability properties, conforming to the shape of the body part without the need to apply excessive pressure. The first layer 5 provides a comfortable wearing against the skin, and prevents adhesion to the skin and/or hair by the core layer 10. The second layer has been found to reduce hardening time and prevents the core 10 from adhering to itself or other articles.

The invention also provides an inflatable cushion 300 made at least partly from the thermoplastic sheet.

The invention also provides a device for molding a sheet 100 prior to medical treatment, and/or for immobilising a subject during medical treatment, especially radiotherapy for instance to the head and neck, comprising a frame 200 that is configured to immobilise and support a sheet of material 100 or cushion 300 over an opening, which opening is dimensioned to receive the impression of a body part.

Material

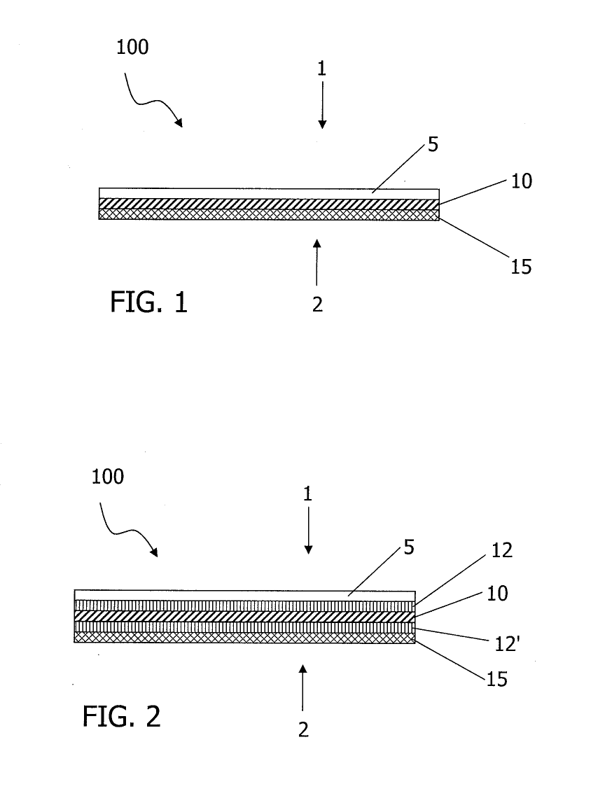

The present invention provides a thermoplastic sheet material deformable under the application of heat to conform to the contours of a portion (e.g. head and neck) of a subject, which after cooling down retains the deformed shape and becomes rigid or semi-rigid. With reference to FIG. 1 the sheet material 100 comprises at least three separate layers—a core layer 10 having an first 1 (skin facing) surface and lower surface 2, a first outer layer 5 disposed over the first 1 surface of the core layer and a second outer layer 15 disposed over the second 2 surface of the core layer—which layers are bonded so as to form a single sheet 100. The overall thickness of the sheet 100 is 1.5 mm, 1.6 mm, 1.7 mm, 1.8 mm, 1.9 mm, 2.0 mm, 2.2 mm, 2.4 mm, 2.5 mm, 2.6 mm, 2.7 mm, 2.8 mm, 2.9 mm, 3.0 mm, 3.2 mm, 3.4 mm, 3.5 mm, 3.6 mm, 3.7 mm, 3.8 mm, 3.9 mm, 4.0 mm depending on the which part of the body needs to be immobilized.

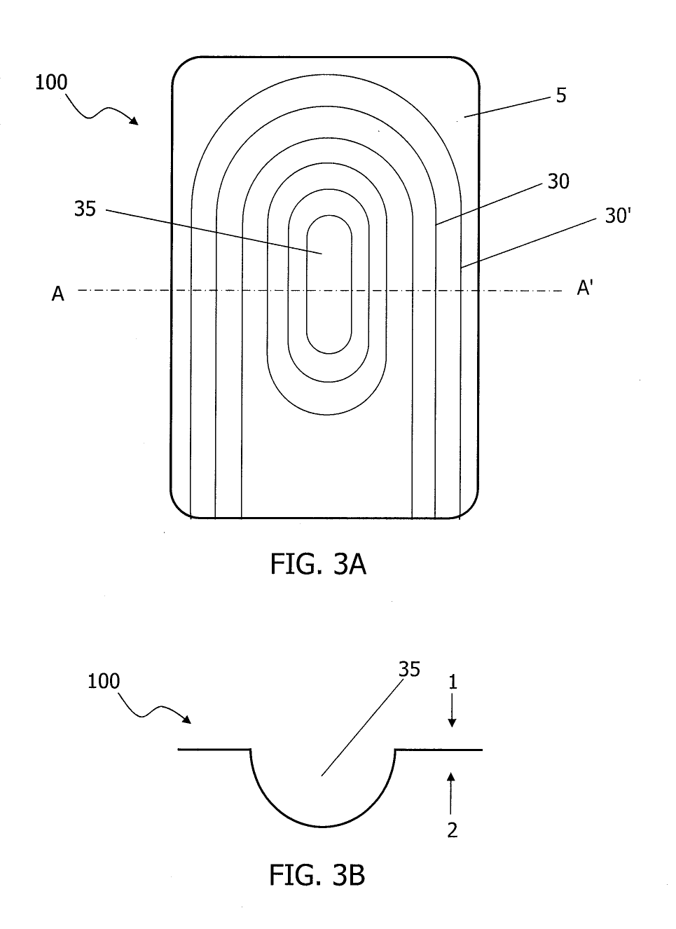

The sheet 100 is distortable at temperatures of between 50 and 70 deg Celsius, depending on the polycaprolactone content. Typically the sheet is brought into the malleable condition by heating in a water bath set around 5 degrees Celsius above the melting temperature, most preferable at 65 deg Celsius. An impression of the body part is taken, for example of the back of the head, by placing the first surface 1 i.e. the first outer layer 5 in contact with the skin and/or hair, and applying pressure to the sheet 100. After molding, the sheet 100 is allowed to cool, hardening in the process. A resulting mask is shown in FIGS. 3A and 3B. In FIG. 3A, the impression of the body part is illustrated as a series of contour lines 30, 30′ which indicate increasing depths from the edges of the sheet 30, 30′ towards a central deepest region 25. A cross-section along line A-A′ is shown in FIG. 3B.

The sheet 100 exhibits excellent deformability properties, conforming to the shape of the body part without the need to apply excessive pressure. The first layer 5 provides a comfortable wearing against the skin, and prevents adhesion to the skin and/or hair by the core layer 10. It also insulates the heat emitted by the heated core 10 layer from the skin of the subject. The second layer 15 prevents the core 10 from adhering to itself or other articles, and in addition reduces the hardening time. Further, the multi-layer sheets can be rolled in the deformed condition without damage to the integrity of the structure. The sheet used for the support of the head, being considerably thinner than conventional masks is semi-rigid, has a soft touch feeling, and, therefore, provides a high degree of comfort in both molding and wearing.

Core Layer

The core layer 10 comprises a thermoplastic composition containing polycaprolactone and polyurethane and having a thickness between 1 mm, 1.5 mm, 2.0 mm, 2.5 mm, 3 mm or a value in the range between any two of the aforementioned values, preferably between 1 mm 3 mm.

The polyurethane may present in an amount of 0%, 10%, 20%, 30%, 40% or 50% (w/w), or a value in the range between any two of the aforementioned values, preferably 20 to 40%, most preferably 30%. The polycaprolactone may be present in an amount of 60%, 70%, 80%, 90% or 100% (w/w) or a value in the range between any two of the aforementioned values, preferably 60 to 80% (w/w), most preferably 70%. Typically, there will be more polycaprolactone than polyuret hane which polycaprolactone lowers the temperature at which the sheet deforms. The ratio of polycaprolactone:polyurethane is (w:w) may be 5:1, 4:1, 3:2, 3:1, 2.3:1, 2:1 preferably 2.3:1.

The molecular weight of the polyurethane may be equal to or less than 10 000, 20 000, 30 000, 40 000, 50 000, 60 000, 70 000, 80 000, 90 000, 100 000, 120 000, 140 000, 150 000, or a value in the range between any two of the aforementioned values, preferably between 10,000 and 100,000. Polyester polyurethane is the preferred polyurethane.

The molecular weight of the polycaprolactone may be 10 000, 20 000, 30 000, 40 000, 50 000, 60 000, 70 000, 80 000, 100 000, 200 000, 300 000, 400 000, 500 000, or a value in the range between any two of the aforementioned values, preferably between 10 000 and 60 000, more preferably between 37 000 and 500 000.

Caprolactone polyester polyurethane is particularly suitable, which polyurethane may be obtained by reacting isocyanate and polycaprolactone-based polyester. Such a caprolactone polyester polyurethane is commercially available as a granulate. The melting point of said polycaprolactone polyester polyurethane lies between 190 and 210 degrees Celsius. By adding the polycaprolactone, also preferably in granulate form, a thermoplastic composition is obtained that is distortable and kneadable at a temperature of ˜69 degrees Celsius and remains distortable by cooling down about 50 degrees Celsius. At this temperature, the core layer 10 may be stretched at least up to twenty times the original length thereof. In the hardened condition, the thermoplastic composition is semi-rigid, unlike a typical rigid face mask, and has a memory effect that, after heating, returns to the shape formed on cooling. It is non-elastic in the hardened condition. The sheet 100 is able to secure the body part of the subject particularly when it is attached around the edges to a frame as described later below.

In an advantageous embodiment of the invention, the core layer 10 comprises between 1 to 40% (w/w) of microspheres of non-metallic, heat-accumulating material which is especially suited for heating in a micro-wave oven. Preferred are glass microspheres with a diameter between 20 and 800 micrometer. A colouring agent may be added to the core.

Intervening Layers

One or more intervening layers 12, 12′ (FIG. 2) may be disposed between the core layer 10 and the first outer layer 5, and/or disposed between the core layer 10 and the second outer layer 15.

Typically an intervening layer 12, 12′ comprises a higher polycaprolactone content compared with the core layer 10, and thus can be deformed at a lower temperature than the core layer 10. According to one aspect of the invention, an intervening layer 12, 12′ has a polycaprolactone content that is higher than the core layer 10 by 5%, 10%, 15%, 20%, 25%, 30%, 50%, 100%, 200%, 300%, or 400% or a value in the range between any two of the aforementioned values, preferably between 100% and 400%. An intervening layer 12, 12′ may be comprised of pure polycaprolactone.

Thickness of an intervening layer 12, 12′ may be 20%, 30%, 40%, 50%, 60%, 70% or 80% that of the core layer 10 or a value in the range between any two of the aforementioned values, preferably between 40% and 60%.

By virtue of an intervening layer 12, 12′, the sheet 100 can be deformed at a lower temperature, since only the only the intervening layer 12, 12′ of the sheet 100 needs to be heated above the temperature at which the thermoplastic material deforms; the result is that heating up time before use is shorter and comfort to the subject is improved. As the core layer 10 has not to be brought at such high temperature, the expansion of the core 10 is also smaller. This is especially advantageous when the sheet 100 is presented in the shape of a roll. Due to expansion when heating, successive windings could be pressed so strongly together than they adhere strongly to each other.

In an advantageous embodiment of the invention, the intervening layer 12, 12′ comprises between 1 to 40% (w/w) of microspheres of non-metallic, heat-accumulating material which is especially suited for heating in a micro-wave oven. Preferred are glass microspheres with a diameter between 20 and 800 micrometer. A colouring agent may be added to the intervening layer 12, 12′.

First Outer Layer

The first outer layer 5, also referred to as the ‘first layer’ herein, comprises a polyamide-based knitted fabric material with a thickness outside the core layer 10 of between 0.05 and 1.5 mm. According to one aspect of the invention, the knitted fabric material is formed from a yarn comprising between 80% to 95% polyamide, and between 5% and 15% elastane, preferably comprising 90% polyamide and 10% elastane. The fabric weight may be 190 g/m², 200 g/m², 210 g/m², 220 g/m², 230 g/m², or 240 g/m², preferably between 210 g/m² and 230 g/m². The fabric may be coloured, for example, with a calming colour such as a neutral tone, pastel shade, or primary colour such as blue.

The thickness of the first outer layer 5 is equal to or less than 0.05 mm, 0.06 mm, 0.08 mm, 0.1 mm, 0.5 mm, 0.8 mm, 1 mm, 1.2 mm, 1.5 mm or a value in the range between two of the aforementioned values, preferably between 0.05 and 1.5 mm, more preferably having a thickness of between 0.1 and 0.4 mm.

The first layer 5 is bonded to the core 10 or intermediate 12, 12′ layer by virtue of the adhesive property of the core 10 in the fluid condition. Adhesion may be enhanced pressing the first layer 5 onto the core layer 10, for example, by the use of roller during manufacture.

Due to the presence of first layer 5, the sheet 100 of the invention may be applied directly in distortable condition on the skin. It does not adhere to hair and skin. It does not either leave visible fingerprints, so that disposable gloves not necessary for the application. In addition, the first layer 5 forms a thin insulating layer, such that the skin is not subject to elevated temperatures that might otherwise harm such areas.

Second Outer Layer

The second layer 15, also referred to as the ‘second layer’ herein, comprises a soft resilient open cell foam plastic disposed over the core layer 10 and forming contact therewith or with the optional intermediate layer 12, 12′.

The second layer 15 forms a physical and insulating layer which protects the core layer 10 in the malleable condition. The foam plastic outside the core layer 10 or optional intermediate layers 12, 12′, before the application, does not comprise thermoplastic material as for example the foam plastic from the composite material according to U.S. Pat. No. 3,728,206 which foam plastic does not form a coating. The second layer 15 forms a covering which prevents portions of the sheet 100 from adhering inadvertently to one another. This allows the sheet to be transported in a deformable condition, rolled up, for example. The second layer 15 allows the sheet to be handled with bare hands without, damage to the core 10 or optional intermediate layers 12, 12′, and without adhering to the fingers.

The thickness of the second outer layer 15 is equal to or less than 0.05 mm, 0.06 mm, 0.08 mm, 0.1 mm, 0.5 mm, 0.8 mm, 1 mm, 1.2 mm, 1.5 mm or a value in the range between two of the aforementioned values, preferably between 0.05 and 1.5 mm, more preferably having a thickness of between 0.4 and 0.6 mm. It has been found that layers of foam plastic with a thickness outside of the core of more than 1.5 mm would prevent a good adherence to the underlying layer (i.e. core or intermediate layer) when the sheet is deformed or rolled up.

When the sheet is presented in the shape of a roll, the maximum thickness of the second layer 15 is preferably of 0.6 mm and when the material is used as a small strip for winding around body parts the thickness of the second layer 15 is preferably not higher than 0.4 mm.

The second layer 15 is made from a material able to withstand temperatures at which the core is softened or weakened. They are preferably made from a non-thermoplastic plastic, such as polyurethane formed into soft, open cell foam. The foam that has such an open-cell structure that core layer 10 in softened condition can traverse it when pressure is exerted but also that the second layer 15 can be elastically deformed without tearing. When the sheet 100 is heated, it can be stretched up to four times its original length without breakage of the second layer 15. Suitable foam plastics for the second layer 15 are polyurethane, particularly polyester polyurethane and polyether foam.

The second layer 15 is bonded to the core 10 or intermediate 12, 12′ layer by virtue of the adhesive property of the core 10. Adhesion may be enhanced pressing the second layer 15 onto the core layer 10, for example, by the use of roller during manufacture.

For some applications, the second layer 15 may be provided with perforations (not shown) with a diameter of at least 0.5 mm and for example 2 mm. Such perforations assist, for example, with heating the core layer 10 by providing access directly to the core, and with drainage when heating the sheet occurs in a warm water bath.

Cross-wise through core 10, first 5 and second 15 layers, smaller perforations with a diameter of at least 0.5 mm and preferably about 1 to 1.3 mm may be provided, so as not to hamper the skin breathing after applying the sheet. Said perforations may lie on rows crossing each other under 90 degrees and making an angle of 45 degrees with the transversal direction of the strip, plate or roll of composite material, at a distance of each other in the rows of 1.5 to 4 mm. The skin may still breathe even after application of the material.

Attachment Means

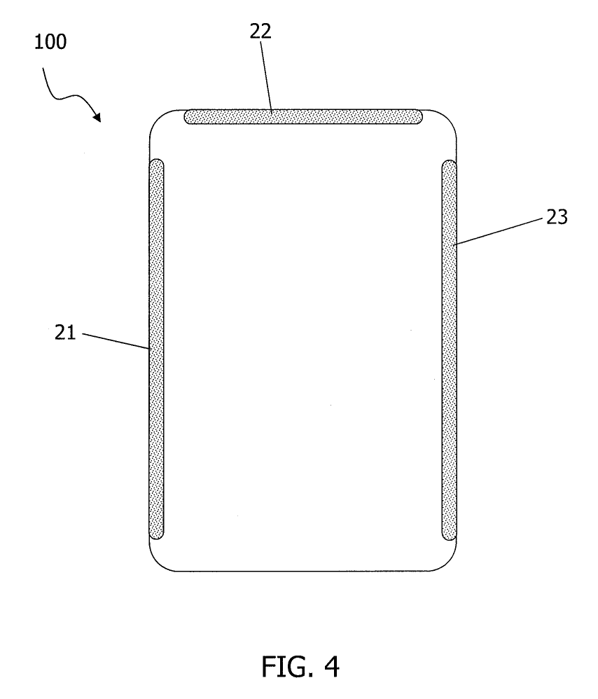

The sheet 100 for use with a frame 200 of the invention discussed below, may further comprise one or more attachment means 21, 22, 23 (FIG. 4), configured to maintain the sheet 100 in position over the opening 56 provided by the frame 200. The attachment means 21, 22, 23 is able to maintain the position of the sheet 100 when it supports the weight of the body portion (e.g. head) during treatment and when the sheet 100 receives the usual pressure applied to the head during its molding. The attachment means 21, 22, 23 preferably cooperates with a corresponding attachment means in the frame 200. The attachment means 21, 22, 23 preferably is connected with at least two opposing edges of the sheet. According to one aspect of the invention, the attachment means 21, 22, 23 comprises a foldable edge, one part of a hook and loop fastener (e.g. Velcro strip), or one part of a zip fastener.

Inflatable Cushion for Use in Medical Treatment

One embodiment of the invention is an inflatable cushion 300 for use in medical treatments as describe above, at least partly made from the thermoplastic sheet 100 of the invention. The cushion 300 comprises a first 110 (e.g. upper) flexible and a second 115 (e.g. lower) flexible sheet that are sealed together, preferably around the edges so as form an inflatable cushion disposed with a lumen 117 in which inflation medium may be held. The first 110 and second 115 flexible sheets are impermeable to inflation medium. Where provided for the molding to and support of a body part, the cushion 300 is dimensioned to receive the body portion (e.g. the head) on the first sheet 115.

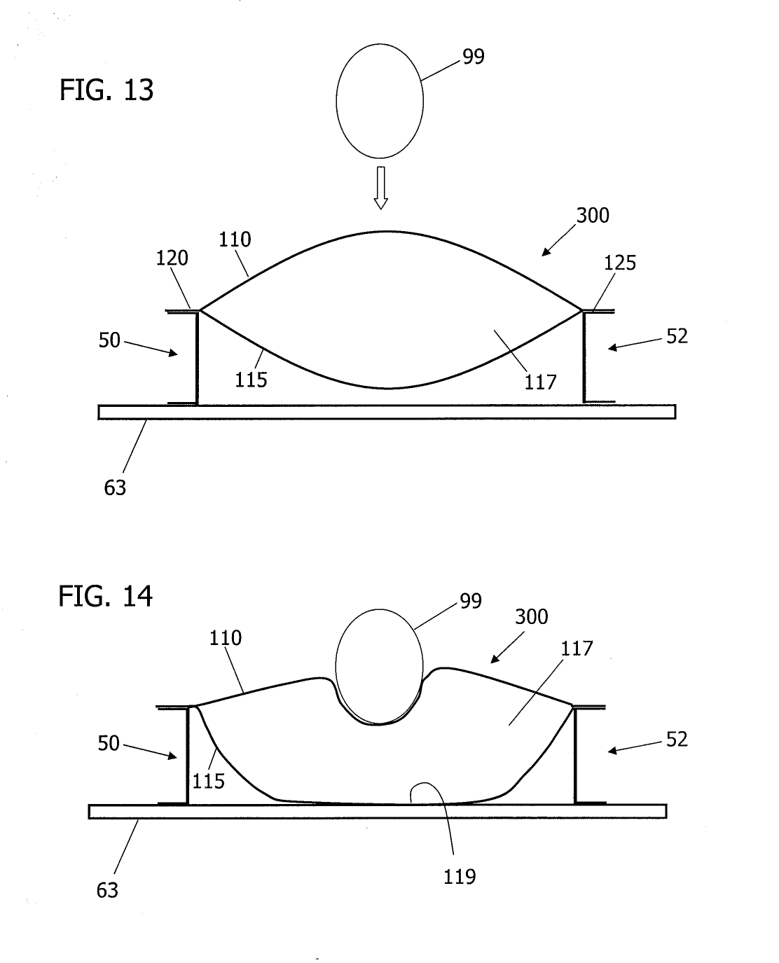

The first flexible surface 115 is at least partially, preferably entirely, formed from the thermoplastic sheet 100 of the invention, and thus is moldable in the heated and deformable condition to fit the contours of a body part applied thereto when the cushion 300 is in the inflated state. The first layer 5 of the thermoplastic sheet 100 faces outwards, while the second layer 15 faces the lumen 117 of the cushion 300. Advantageously, inflation introduces a gap between the first flexible sheet 110 and the second flexible sheet 115 which allows the cushion 300 to be placed on a flattened surface such as a table during molding and prevents the first flexible sheet 110 from contacting the flattened surface while the body part is pressed onto the cushion, which would otherwise result in a false impression e.g. in a flattened plateau where both sheets 110, 115 touch the table, said impression not representative of the body part. Moreover, because of the medium inside, the cushion will automatically fit itself anatomically to the object, for example, to the back of the head and the neck. FIGS. 13 and 14, showing transverse cross sections of a cushion 300 of the invention attached to table 63—mounted longitudinal supports 50, 52 of a frame (described below) via seams 120, 125, illustrate the sequence of taking an impression of a body part 99. In FIG. 13, the cushion 300 in a deformable (heated) condition is inflated, and the body part 99 applied thereto. To deform the cushion, it is typically heated in a water bath at 65 deg C. In FIG. 14, the inflated cushion adapts to the shape of the body part. While the second sheet 115 of the cushion contacts the table 63 and forms a plateau 119, the first sheet 110 remains true to the shape of the body part 99 being prevented from contacting the table 63 by virtue of the medium filled lumen 117. In other words, no false tension is obtained due to the fact that the part of the body 99 will not touch any additional support. During hardening, the pressure or volume of medium in the lumen can be adjusted in order to improve the shape of the impression. For example, medium can be added to or released from the lumen as the cushion cools. Once the first sheet 110 of the cushion has hardened, the cushion 300 may subsequently be used to immobilise the body portion in radiotherapy or other treatments. Hardening time for a cushion formed from sheet having a 2 mm thickness of 2 mm is typically 3 minutes. After treatment, the cushion 300 may be remolded to fit another patient by reheating the thermoplastic sheet.

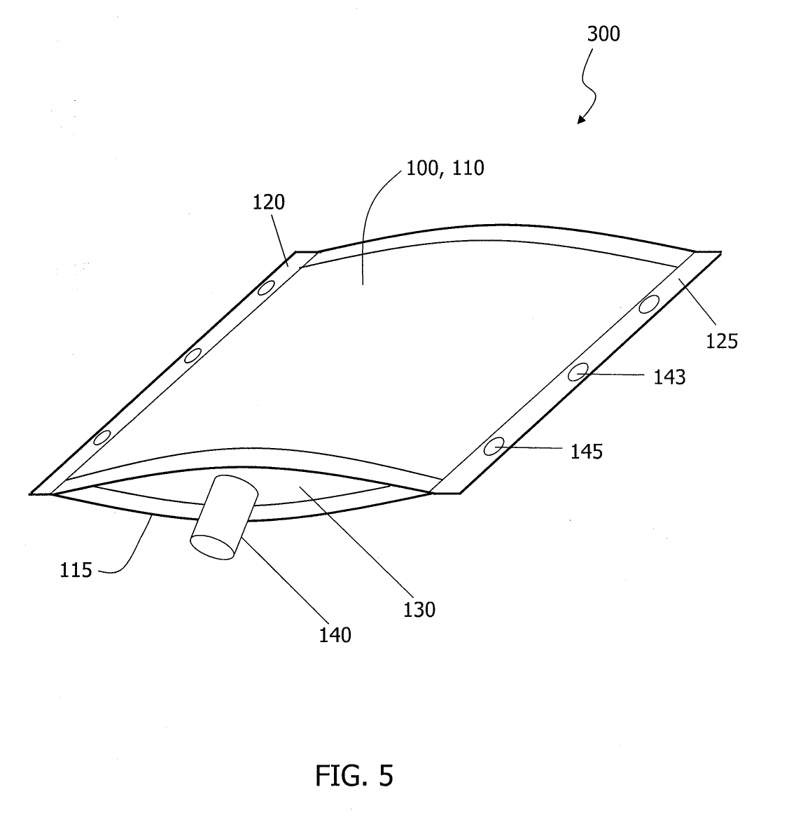

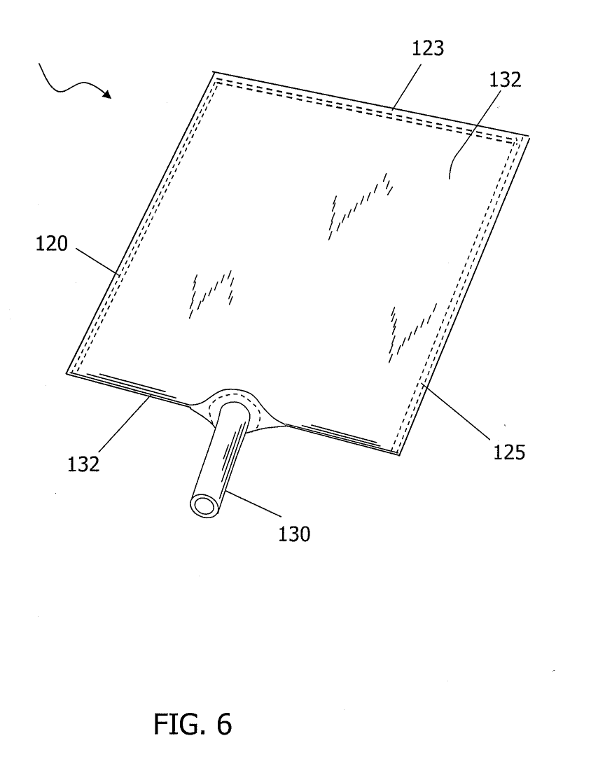

With reference to FIGS. 5 and 6 inflatable cushion 300 of the invention comprises a first flexible sheet 110 formed from the thermoplastic sheet 100 of the invention, and a second flexible sheet 115 formed from a flexible material, that may be another thermoplastic sheet 100 of the invention or made from a heat resistive flexible material such as polyurethane. Preferably, the second flexible sheet 115 should not exhibit elastic properties. Both sheets, placed one over the other such that the second outer layers 15 are facing each other, and are sealed to form an inflation lumen 117, typically by a sealing seam (e.g. 120, 125) around the edges, for example, using adhesive, ultrasonic or heat sealing, or by the use of clamps. In a variation, shown in FIG. 5 one or more side pleats 130 may be introduced between the first 110 and second 115 sheets that increase separation between the first 110 and second 115 flexible sheets after inflation. A side pleat 130 may be made from thermoplastic sheet 100 or from a heat resistive flexible material such as polyurethane. The nozzle is preferably attached using adhesive. FIG. 5 depicts an instance of the invention having one side pleat 130 onto which the nozzle 140 is affixed.

In a preferred embodiment of the invention, and as shown in FIG. 6 the cushion 300 is formed from a single thermoplastic sheet 100 of the invention, folded and sealed (120, 123, 125) around the remaining three edges to form the inflation lumen 117. The folded edge 132 is disposed with the inflation nozzle 140.

The lumen 117 of the cushion 300 is in fluid connection with an inflation nozzle 140 configured to permit passage of an inflation medium which may be gaseous (e.g. air, oxygen, nitrogen) or liquid (e.g. saline or water). The inflation nozzle 140 may be disposed with a one way valve which prevents inflation medium from leaving the inflation lumen 117. The valve may have a release mechanism that allows inflation medium to pass out from the lumen 117, for example, by a pinching action. Alternatively, or in addition, the inflation nozzle 140 may be disposed with sealing cap. The inflation nozzle 140 is attached to the cushion 300 in a location that does not interfere with taking the impression, for example, at the edge of the cushion 300 as depicted in the figures. It should also be located so as to avoid interference with the attachment means (see below). In the case of a cushion for taking an impression of the head and neck, the nozzle is preferably located at the head end of the cushion.

The cushion 300 for use with a frame 200 of the invention discussed below, may further comprise one or more attachment means, configured to maintain the cushion 300 in position over an opening 56 provided by the frame 200. The attachment means is configured to maintain the position of the cushion 300 when it supports the weight of the body portion (e.g. head) during treatment and when the cushion 300 receives the usual pressure applied to the body portion during molding. The attachment means cooperates with a corresponding attachment means in the frame, or co-operates with an intermediary buffering structure that connects the cushion attachment means with the frame attachment means. The attachment means is preferably disposed along two opposing edges of the cushion 300. According to one aspect of the invention, the attachment means comprises one or more apertures 140, 143 located in a seam 125 of the cushion 300. According to another aspect of the invention, the attachment means comprises opposing seams 120, 125 of the cushion 300; which provide edges that can be clamped onto the frame.

Buffering Structure

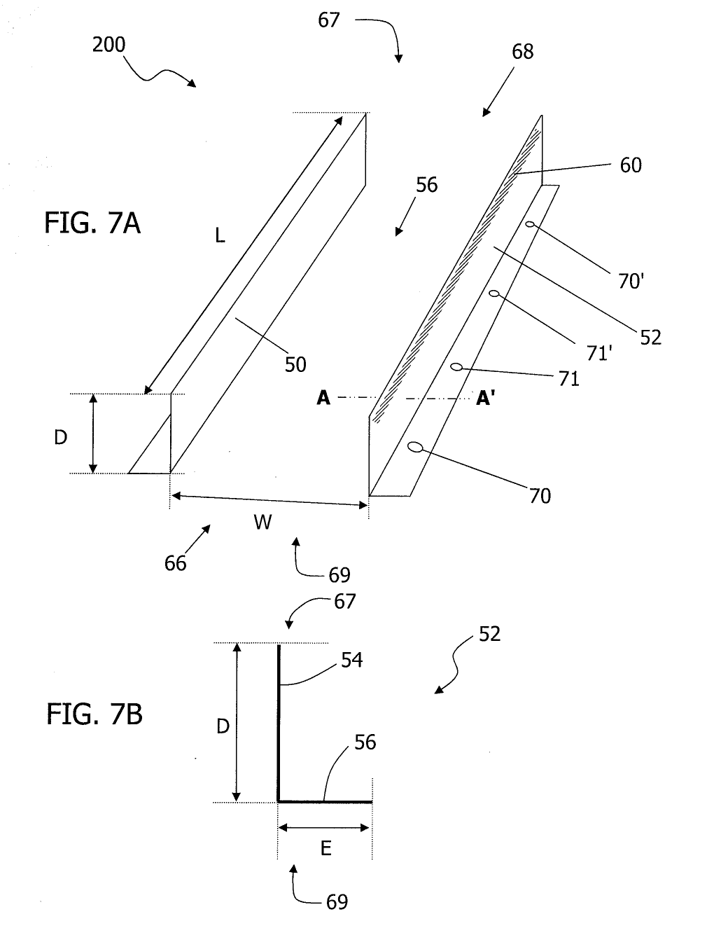

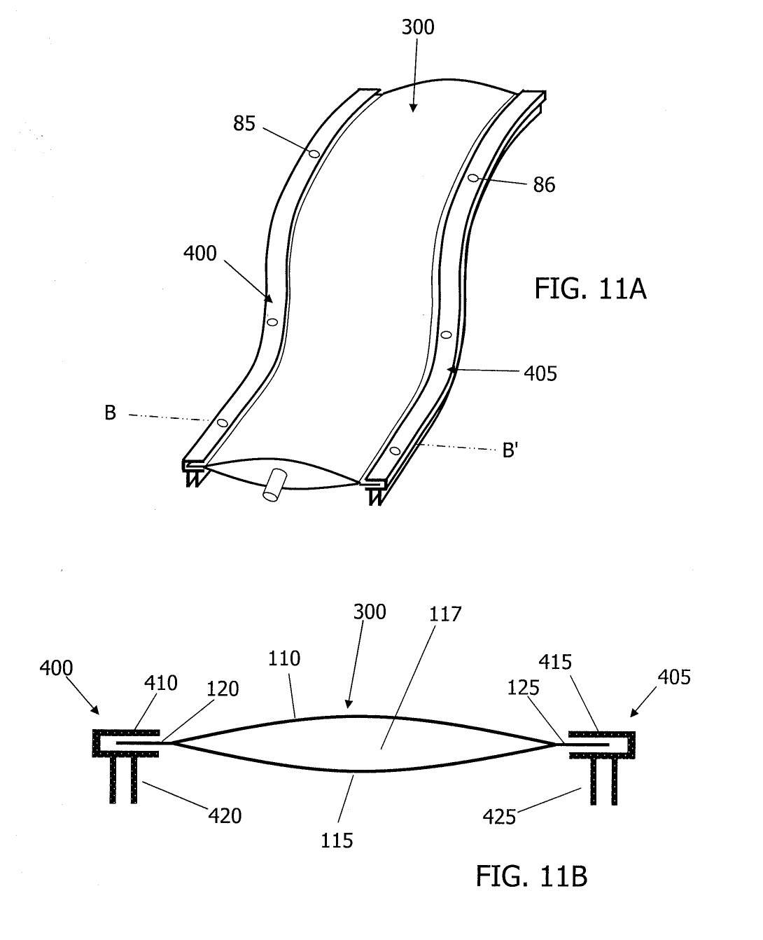

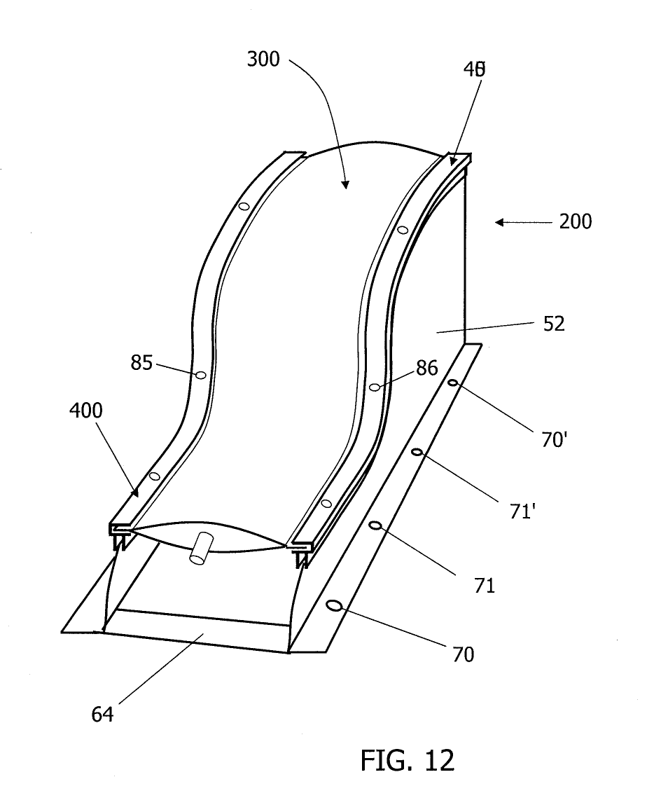

The buffering structure of the invention is a structure that demountably attaches the cushion 300 to the frame 200 and maintains the cushion in a rigid position with respect to the frame. With reference to FIGS. 11B, 15 and 16 the buffering structure 400, 405 comprises a cushion attachment element 410, 415, that receives and attaches the cushion 300, and a frame attachment element 420, 425 that receives and attaches the frame 200 preferably to the top of a long part 54 (FIG. 7B) of a longitudinal support 50, 52. The cushion attachment element 410, 415 is rigidly fixed to the frame attachment element 420, 425 maintaining a constant juxtaposition between the frame 200 and cushion 300. A buffering structure 400, 405 of the invention is preferably formed from an elongate structure of rigid material, comprising cushion attachment element 410, 415 which is first elongated groove configured to receive the seam 120, 125 of a cushion 300. The frame attachment element 420, 425 according to one embodiment, is a second elongated groove configured to receive an edge of the frame 200 and the first groove is preferably perpendicular to the second groove as shown in FIG. 11B. The buffering structure may be provided with one or more apertures 85, 86 for insertion of securing pins which disengagably connect with the cushion seam.

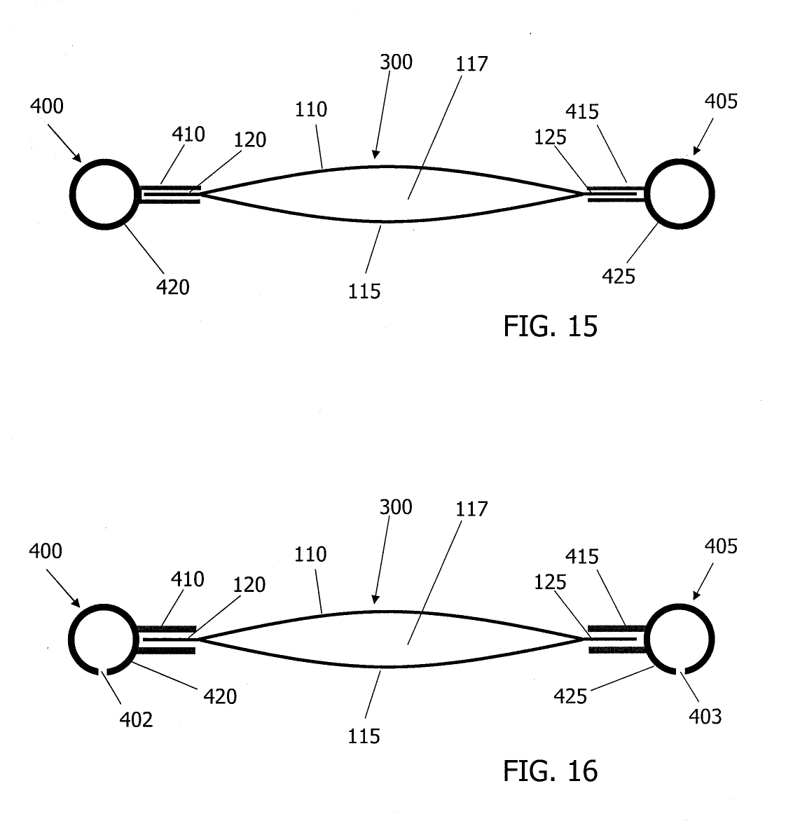

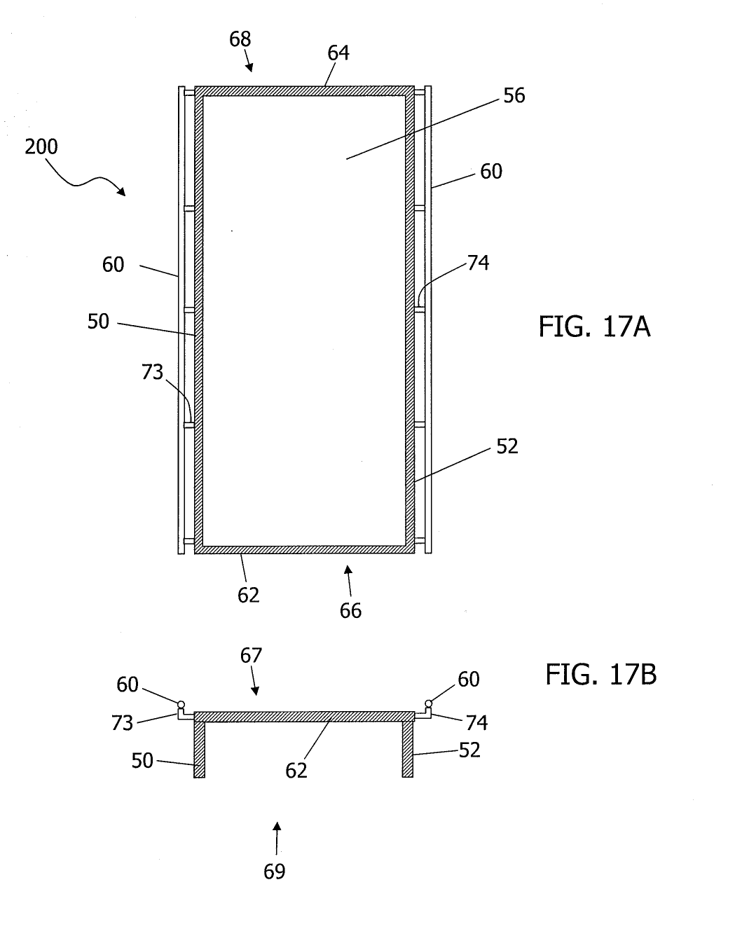

In an alternative embodiment shown in FIG. 15, the frame attachment element 420, 425 is a hollow, longitudinal, tubular support, configured to receive an elongate cylindrical support rail extending the longitudinal length of the frame 200 (see, for example, 60, FIG. 17A, 17B). In variation shown in FIG. 16, the frame attachment element 420, 425 is a hollow, longitudinal, tubular support disposed with a longitudinal slit 402, 403, configured to receive an elongate rail element extending the longitudinal length of the frame 200. The slit 402, 403 is configured to pass over any structural members (see, for example, 73, 74, FIG. 17A, 17B) used to affix the rail to the frame.

Molding Frame

The invention also provides a device for molding a sheet 100 or cushion prior to medical treatment, and for immobilising a subject during medical treatment, especially radiotherapy to the head and neck, comprising a molding frame 200 that is configured to immobilise and support either a sheet of material 100 or the cushion 300 of the invention over an opening, which opening is dimensioned to receive the impression of a body part.

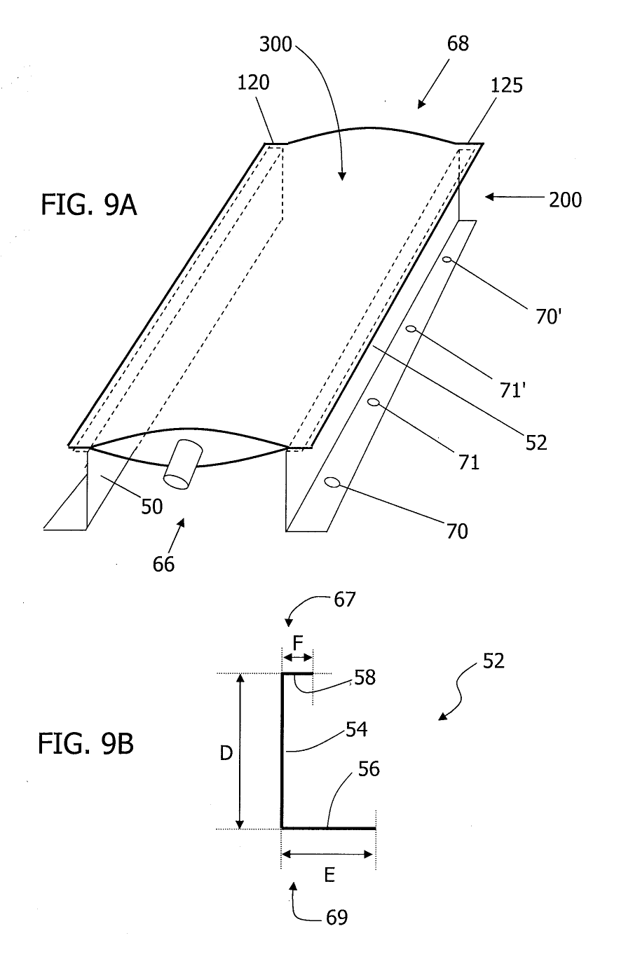

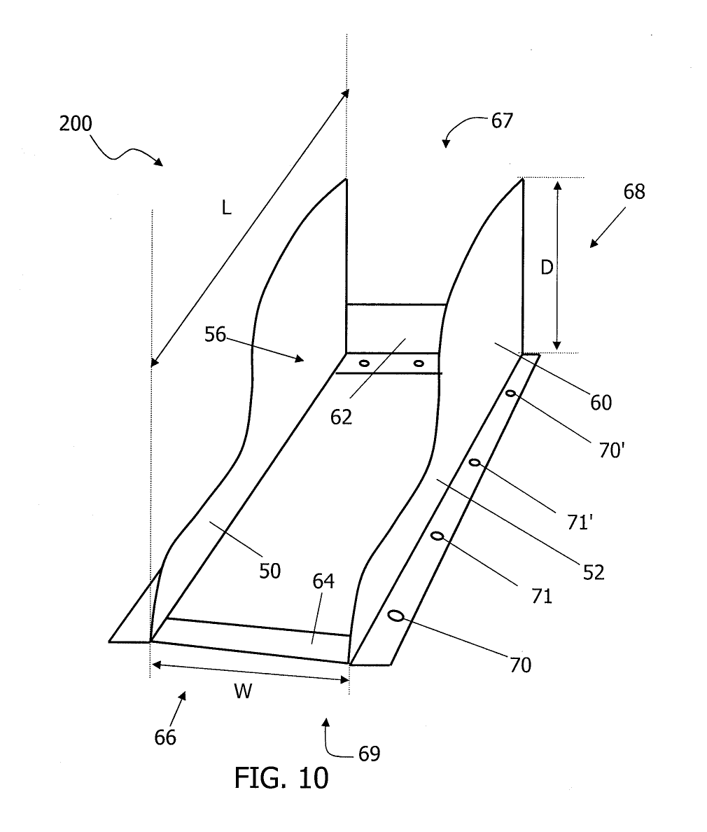

With reference to FIGS. 7A and 10, the molding frame 200 having a top end 67 and a base end 69, comprises two rigid longitudinal supports 50, 52 arranged opposite each other, which flank an opening 56 configured to receive a sheet 100 or cushion 300 of the invention over the top 67 of the opening 56. The frame 200 has an at least partially open first end 66 and an at least partially open second end 68 opposite thereto which allow access to the opening 56 when the top 67 of the frame is disposed with a sheet 100 or cushion 300. As shown in FIG. 9, the frame may be disposed with one or more cross-supports 62, 64 that rigidly link and separate the longitudinal supports 50, 52. Where present, the cross supports will also flank the opening 56 i.e. be disposed at the ends 66, 68 of the frame. However, such cross-supports 62, 64 are not essential since the longitudinal supports 50, 52 may be separated by rigid attachment to the treatment table, to a rigid plate that couples to the treatment table, or to another rigidity providing means.

The supports 50, 52 comprise one or more securing means 70, 70′ at the base end adapted for demountable attachment to a treatment table or to a rigid plate that couples to the treatment table, or to other fixing means. The securing means 70, 70′ fixes the supports 50, 52 so they are immobilised relative to the table and to each other. The securing means 70, 70′ may comprise one or more apertures that pass through the supports 50, 52. The aperture can receive a bolt or pin that affixes the frame to treatment table or to a rigid plate provided with a reciprocating aperture. Once fixed, the supports 50, 52 cannot move without undue force, and are able to support the weight of the body part during treatment and also any usual pressure applied thereto during molding of the sheet 100. After treatment, the supports 50, 52 can be demounted from the treatment table or from the rigid plate. The supports 50, 52 may further comprise additional securing means 71, 71′ that can receive and demountably secure a face mask. These additional securing means 71, 71′ may also be apertures that pass through the supports 50, 52. Such additional aperture can receive a bolt or pin that affixes the face mask to the frame and optionally also to the treatment table or to a rigid plate.

The opening 56 formed by the frame 200 is dimensioned to receive the body portion (e.g. head) or more preferably, an impression thereof. The length, L, of the opening defined by the length of the longitudinal supports 50, 52, and width, W, of the opening 56 (FIGS. 7A and 10) between the longitudinal supports 50, 52 will be determined in part by the dimensions of the treatment table, but general will be large enough to receive, for example, the head. The depth, D, between the top 67 and the base 69 of the opening 56 is sufficient to accommodate the body part, and to allow a gap between the deepest depression formed in the sheet 100 and the base of the aperture 56.

Advantageously, the gap provides a route for excess water to drain from the sheet 100 after it has been retrieved from a water bath during deformation. The gap also provides a cooling channel for the circulation of air that assists rapid hardening.

It will be appreciated that the dimensions of the frame 200 will vary according to the body portion and on the size of the subject, however, as a general guidance, the dimensions of a frame to accommodate the shape of a head, may have a length, L, that is at most 25 cm, 30 cm, 35 cm, 40 cm, 45 cm, or 50 cm, or a value in the range between any two of the aforementioned values, preferably between 25 and 40 cm. The width W, may be at most 12 cm, 14 cm, 16 cm, 18 cm, 20 cm, 22 cm, 24 cm, 36 cm, 48 cm, 30 cm, or a value in the range between any two of the aforementioned values, preferably between 12 and 24 cm. The depth D, may be at most 4 cm, 6 cm, 8 cm, 10 cm, 12 cm, 14 cm, 16 cm, or 18 cm, or a value in the range between any two of the aforementioned values, preferably between 4 and 10 cm.

In a preferred embodiment a longitudinal support 50, 52 comprises an L-shaped profile, the long part of the “L” providing depth, D, to the frame, and the narrow part of the L perpendicular, or adopting an angle between 85 deg and 95 deg to the long part and disposed with the aforementioned securing means 70, 70′, 71, 71′. FIG. 6B illustrates an L-shaped profile of a longitudinal support 50, 52 showing the long part 54 having depth, D, and perpendicular thereto the narrow part which extends from the base 69 of the long part 54 by an amount E. FIG. 9B also illustrates an L-shaped profile of a longitudinal support 50, 52 showing the long part 54 having depth, D, and perpendicular thereto the narrow part which extends from the long part 54 by an amount E, said longitudinal support 50, 52 further comprising a horizontal lip 58 which appears in the profile as an extension from the top 67 end of the long part 54. The lip 58 extends from the long part 54 of the L-shaped profile by an amount F. As seen in FIG. 9A, the lip 58 may provide an attachment means for a cushion of the invention.

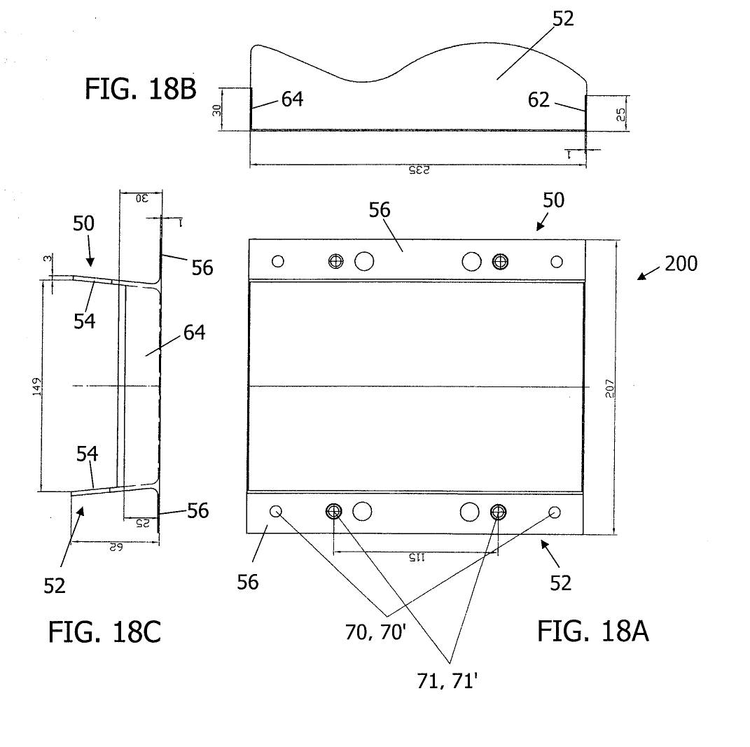

According to a particular embodiment of the frame 200 depicted in FIG. 7A, the depth D of the frame 200 is essentially constant along the length of each support 50, 52. According to another embodiment of the invention, the depth D of the frame 200 may vary along the length of each support 50, 52 which provides a side profile that better accommodates the shape of a body part. FIG. 10 shows an instance of a frame 200 of the invention whereby the depth D of the frame changes as a function of the length; in the illustrated embodiment, the frame is deeper at the second end 68 compared with the first end 66. The depth between the first 66 and second 68 ends may vary in any manner, but preferably in a gradual fashion. According to one embodiment of the invention, the depth D frame follows a path corresponding to a continuous curved line having only one point of inflection, which inflection point defines one convex part and one concave part. A particular embodiment of a frame of the invention is depicted in FIGS. 18A to 18C whereby the side profile is sinusoidal.

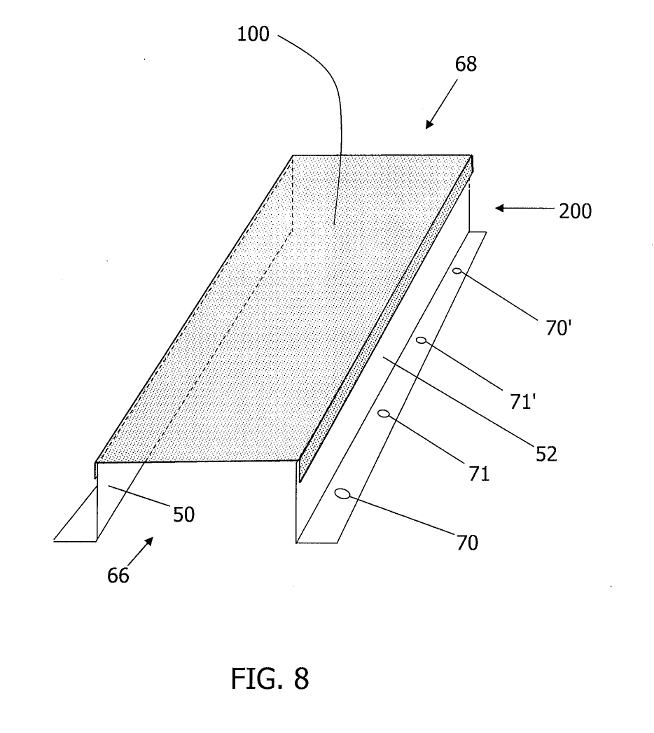

The supports 50, 52 further comprise one or more demountable attachment means 60 for the sheet 100 or cushion 300, configured to maintain the sheet 100 or cushion 300 in position over the opening 56 as shown in FIGS. 8 and 9A. The attachment means 60 is able to maintain the position of the sheet 100 or cushion 300 when it supports the weight of the body portion (e.g. head) during treatment and when the sheet 100 or cushion 300 receives the usual pressure applied to the head during its molding. The attachment means 60 preferably cooperates with the corresponding edge of the sheet 100, or with a seam edge of a cushion, or with a buffering structure 400, 405 attached to the cushion. The attachment means 60 is preferably disposed on the long part 54 of an L-shaped profile support 50, 52. According to one aspect of the invention, the attachment means 60 comprises a spring clip, a screw-clip, one part of a hook and loop fastener (e.g. Velcro strip), or one part of a zip fastener. According to another aspect of the invention, the attachment means is comprised in a lip 58. As mentioned elsewhere, the frame 200 may alternatively be attached to the cushion 300 via a buffering structure 400, 405.

FIGS. 17A and 17B show a particular embodiment of the invention whereby the frame 200 is dimensioned for taking an impression of the entire body i.e. resembles a bed frame. As with the other frames described herein, the bed frame 200 has a top end 67 and a base end 69, comprises two rigid longitudinal supports 50, 52 arranged opposite each other, which flank an opening 56 configured to receive a sheet 100 or cushion 300 of the invention over the top 67 of the opening 56. The frame 200 has an at least partially open first end 66 and an at least partially open second end 68 opposite thereto which allow access to the opening 56 when the top 67 of the frame is disposed with a sheet 100 or cushion 300. The frame is disposed with cross-supports 62, 64 that rigidly link and separate the longitudinal supports 50, 52. Each longitudinal support 50, 52 extends to the floor via one or more legs.

The opening 56 formed by the frame 200 is dimensioned to receive the whole body impression. The length of the opening defined by the length of the longitudinal supports 50, 52, and width of the opening 56 between the longitudinal supports 50, 52 will be determined in part by the dimensions of the subject. The depth, between the top 67 and the base 69 of the opening 56 is sufficient to accommodate the body, and to allow a gap between the deepest depression formed in the sheet 100 and the base of the aperture 56. Each supports 50, 52 further comprise a demountable attachment means 60, that is an elongate rail for a receiving buffering structure 400, 405 as shown in FIG. 16, which rail attaches to the longitudinal support 50, 52 using one or more bridging links 73, 74. The cushion connected to two buffering structures as shown in FIG. 16 attaches to the bed frame along rails.

Inflatable Cushion for Use as a Container

An alternative embodiment of the invention is an inflatable cushion 300 at least partly made from the thermoplastic sheet 100 of the invention, wherein the first outer layer 5 is optional. Such cushion is suitable for non-therapeutic applications, for example, for a moldable medium container wherein the outer surface of the container does not need to provide comfort to the skin. Such container has application in the dead space of vehicles, sailing vessels etc. such as behind paneling, where they may be inflated and provide storage for media such as water and liquid fuel. Advantageously, dead space, normally difficult to access and having an irregular shape, can be utilised fully since the cushion expands to accommodate the otherwise unusable regions, regardless of the shape. The embodiments described above in regard of the construction of a cushion for medical treatments also apply to the cushion for use as an inflatable medium container. Most preferably, the cushion is formed from a single thermoplastic sheet 100 of the invention having an optional first outer layer, folded and sealed around the remaining three edges to form the inflation lumen. The folded edge is disposed with the inflation nozzle.

What is claimed is:

1. A flexible thermoplastic sheet material, suitable for molding to and immobilising a part of a subject during medical treatment, comprising:
a core layer having an upper surface and lower surface, that is a thermoplastic composition comprising polycaprolactone and polyurethane,
a first outer layer disposed over the upper surface of the core layer, comprising a material formed from a yarn comprising polyamide and elastane,
a second outer layer disposed over the lower surface of the core layer comprising open cell foam,
which layers are bonded so as to form a single sheet.

2. Sheet according to claim 1, wherein the core layer comprises 20% to 40%, polyurethane, and 60% to 80% (w/w) polycaprolactone.

3. Sheet according to claim 1, wherein the core layer further comprises between 1 to 40% (w/w) of non-metallic, heat-accumulating microspheres.

4. Sheet according to claim 1, wherein the yarn of the first outer layer comprises between 80% to 95% polyamide, and between 5% and 15% elastane.

5. Sheet according to claim 1, wherein the fabric weight of the first outer layer is between 210 g/m² and 230 g/m².

6. Sheet according to claim 1, wherein the thickness of the first outer layer is between 0.05 and 1.5 mm.

7. Sheet according to claim 1, wherein the second outer layer is made from polyurethane, polyester polyurethane or polyether open-cell foam.

8. Sheet according to claim 1, further comprising an intervening layer disposed between the core layer and the first outer layer, and/or disposed between the core layer and the second outer layer, made from the same material as the core and with a higher polycaprolactone content.

9. Sheet according to claim 1, having a maximum total thickness of 1.5 to 1.7 mm.

10. An inflatable cushion dimensioned to receive a body portion comprising
a first flexible sheet made at least partially from a thermoplastic sheet according to claim 1,
a second flexible sheet, optionally made at least partially from a thermoplastic sheet according to claim 1,
which sheets are sealed together so as form a lumen that can receive and retain inflation medium, and
an inflation nozzle for the passage of inflation medium to the lumen.

11. Inflatable cushion according to claim 10, further comprising one or more side pleats between the first and second flexible sheets.

12. Inflatable cushion according to claim 10, further comprising an attachment means for attachment of the cushion to a molding frame.

13. Inflatable cushion formed from a single piece of thermoplastic sheet according to claim 1, folded and sealed so as form a lumen that can receive and retain inflation medium, and
an inflation nozzle for the passage of inflation medium to the lumen.

14. An inflatable cushion comprising
a first flexible sheet made at least partially from a sheet according to claim 1 wherein the first outer layer is optional,
a second flexible sheet, optionally made at least partially from a sheet according to claim 1 wherein the first outer layer is optional,
which sheets are sealed together so as form a lumen that can receive and retain inflation medium, and
an inflation nozzle for the passage of inflation medium to the lumen.

15. Inflatable cushion according to claim 14, further comprising one or more side pleats between the first and second flexible sheets.

16. Inflatable cushion formed from a single piece of thermoplastic sheet according to claim 1, wherein the first outer layer is optional, folded and sealed so as form a lumen that can receive and retain inflation medium, and
an inflation nozzle for the passage of inflation medium to the lumen.
